(19) United States
(12) Patent Application Publication
(10) Pub. No.: US 2024/0415086 A1
(43) Pub. Date: Dec. 19, 2024

(54) MARIGOLD MALE INBRED LINE DENOMINATED KI3928

(71) Applicant: KEMIN INDUSTRIES, INC., Des Moines, IA (US)

(72) Inventors: Arvind H. HIRANI, West Des Moines, IA (US); Justin COX, Canyon, TX (US); John A. Greaves, Johnston, IA (US)

(73) Assignee: KEMIN INDUSTRIES, INC., Des Moines, IA (US)

(21) Appl. No.: 18/742,754

(22) Filed: Jun. 13, 2024

Related U.S. Application Data
(60) Provisional application No. 63/472,707, filed on Jun. 13, 2023.

(51) Int. Cl. A01H 6/14; A01H 5/02

(52) U.S. Cl. CPC A01H 6/1496; A01H 5/02

(57) ABSTRACT

A new and distinct inbred plant of Tagetes erecta named KI3928 and characterized by elevated levels of lutein in hybrids and fertile male flowers with desirable pollen shading characteristics.

### CROSS-REFERENCE TO RELATED APPLICATIONS

The present application claims the benefit of priority to U.S. Provisional Patent Application No. 63/472,707, filed Jun. 13, 2023, entitled “MARIGOLD MALE INBRED LINE DENOMINATED KI3928,” the entire disclosure of which is incorporated by reference in its entirety.

### FIELD OF THE INVENTION

The present invention relates generally to marigolds (Tagetes erecta) and, more specifically, to a novel marigold male inbred line denominated KI3928, which has high flower yield and increased levels of lutein on a per acre basis in hybrid combination.

### BACKGROUND OF THE INVENTION

Xanthophylls are yellow pigments that form one of two major divisions of the carotenoid group. Their molecular structure is similar to carotenes, which form the other major carotenoid group division, but xanthophylls contain oxygen atoms, while carotenes are purely hydrocarbons with no oxygen. Xanthophylls contain their oxygen either as hydroxyl groups and/or as pairs of hydrogen atoms that are substituted by oxygen atoms acting as a bridge (epoxide).

Like other carotenoids, xanthophylls are found in the highest quantity in the leaves and/or flowers of most green plants, where they act to modulate light energy. The xanthophylls found in the bodies of animals, and in dietary animal products, are ultimately derived from plant sources in the diet. For example, the yellow color of chicken egg yolks, fat, and skin comes from ingested xanthophylls (primarily lutein, which is often added to chicken feed for this purpose).

The yellow color of the human macula lutea in the retina of the eye comes from the lutein and zeaxanthin it contains; both xanthophylls again requiring a source in the human diet to be present in the eye. These function in eye protection by ionizing blue light, which they absorb.

The group of xanthophylls includes (among many other compounds) lutein, zeaxanthin, neoxanthin, violaxanthin, and α- and β-cryptoxanthin.

There is an interest in developing products that have high levels of xanthophylls. For example, xanthophylls are known to have antioxidant properties and have been shown to prevent age-related macular degeneration (AMD) when present in sufficient quantities in the diet.

Marigold flowers are the most commercially significant source of xanthophylls. Most marigold plants used for the production of xanthopylls are grown from hybrid seed produced by the crossing of a female inbred line and a male inbred line. Accordingly, male and/or female lines of marigold having high levels of xanthophylls can be used to create hybrid seed that can be used to grow hybrid marigold plants with elevated levels of xanthophylls and larger flowers, and thereby improve the economy of producing xanthopylls for the commercial marketplace; particularly if the hybrid plants have a large biomass yield of flowers that can be reliably harvested.

### SUMMARY OF THE INVENTION

The invention consists of a novel marigold (Tagetes erecta) male inbred line, designated KI3928, that has elevated levels of petal lutein content in hybrid combinations, fertile male flowers, excellent vigor, very good pollen shedding characteristics and overall agronomic robustness. KI3928 is a unique marigold male inbred line selected through combination of conventional and molecular breeding to improve (i) petal lutein content in hybrid combinations, (ii) unique assembly of alleles for several QTLs that contribute excellent hybrid/double flower frequency of >90% for several harvests in hybrids; and (iii) very good pollen shedding characteristics that is highly desirable for wind pollination for hybrid seed production. The male inbred line was selected from a purpose-bred population using some of the selection criteria in various generations including petal lutein content on dry weight basis, pollen shedding, flower size, seed set, number of flowers, plant height and uniformity of individual families. Progressive progeny selection therefore increases the petal lutein content of a hybrid by up to 50% with generally good combining ability with female testers.

Plants of the male line cultivar KI3928 have not been observed under all possible environmental conditions. The phenotype may vary somewhat with variations in environment and cultural practices such as temperature, light intensity, day length, water status, and/or fertilizer rate or type without, however, any variance in genotype.

An object of the present invention is a marigold male inbred line which contributes specific sets of alleles in hybrid combinations with a set of female parents that elevates lutein levels for use as an antioxidant in human and animal food, beverages and personal care products. Another object of the invention is a male inbred line of Tagetes erecta that is novel, stable, and uniform and has very good pollen shedding for seed production, and good agronomic characteristics that confers the trait of hyper-accumulation of lutein to its hybrid progeny.

### BRIEF DESCRIPTION OF THE DRAWINGS

FIG. 1 is a scheme of the development process of male inbred line KI3928.

FIG. 2A is a chart comparing the average petal lutein content (PLC) in five hybrids derived from male line KI3928 against a first control in field trials.

FIG. 2B is a chart comparing the average petal lutein content (PLC) in five hybrids derived from male line KI3928 against a second control in field trials.

### DETAILED DESCRIPTION OF THE INVENTION

The present invention relates to a new and distinct inbred plant of Tagetes erecta named KI3928 and characterized by elevated levels of lutein in hybrids and fertile male flowers with desirable pollen shading characteristics.

According to one aspect of the present invention, the novel plant line contributes male fertile flowers with very good pollen shedding characteristics which is greatly suited to hybrid seed production under a wind pollination system. Another advantage of the present invention is the substantial increase in petal lutein content (PLC) on a dry weight basis compared to two commercially grown control hybrids, CK-1 and CK-2. Commercial CKs are the dominant marigold hybrids, commercially cultivated, for flower production for xanthophyll in the commercial growing area.

Another aspect of the present invention relates to excellent general combining ability with a set of female inbred lines for >90% hybrid/double flowers for >6 sequential harvests, that increase overall flower yield.

Another aspect of the present invention relates to the combination of substantial increase in PLC and higher flower yield significantly increasing lutein yield on a per acre basis under ideal growing environments, making these hybrids a more sustainable source of the target carotenoids.

As used herein, the term “F” generation has used herein is the seed (and resulting plants) derived from selected individuals from self-pollination through the single seed decent (SSD) pedigree breeding and selection method. The term “progeny” refers to the plants and seeds of all subsequent generations resulting from a selfed plant from the previous generation.

### Examples—The Development of KI3928

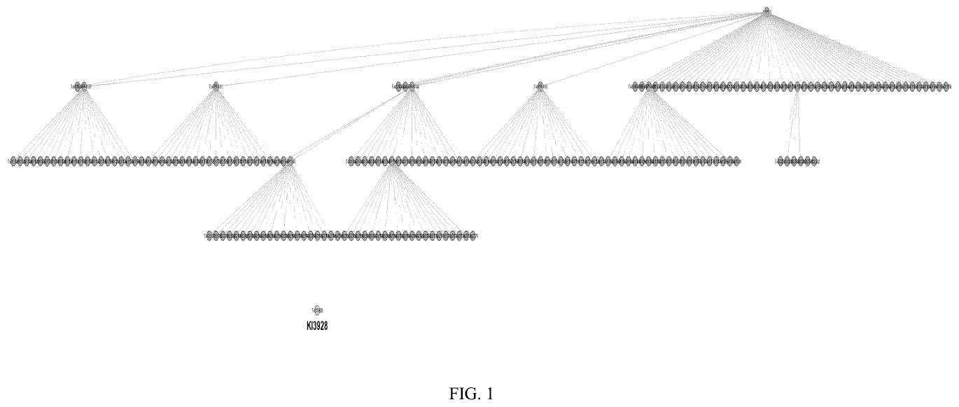

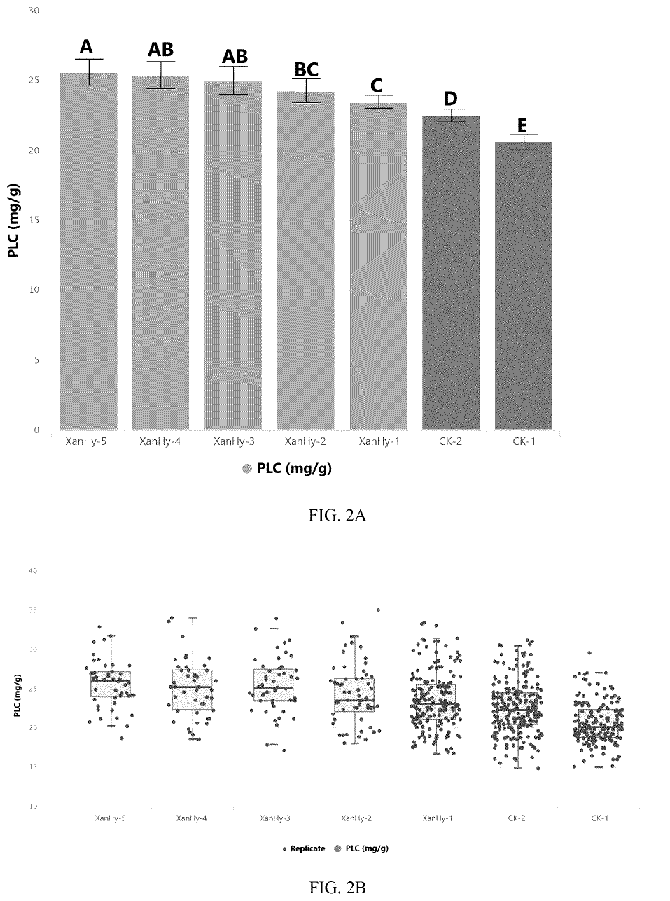

KI3928 was identified as an F₅ plant with dark orange flowers, exhibiting a noticeable increase in petal lutein content in comparison to other plants within the population; and the open pollinated variety, Scarletade. KI3928 was able to pass on this trait to progeny in hybrid combinations. Initial molecular breeding and selection of parents and the F₁ individuals were carried out for selecting desirable traits to drive PLC and double flower frequency in hybrid combinations and to achieve >90% homozygosity in inbred line selection. Subsequently, KI3928 was bred and advanced from the F₂ to F₅ generation using a single-seed descent and half-sib breeding procedure as shown in FIG. 1. Briefly Te009-104 identified as an F₂ individual, was naturally self-pollinated in isolation to produce an F₃ family. Eighty F₃ individuals were generated from F₃ seed and flowers were quantitated for lutein content using a modified AOAC method. All the selfed and half-sib generations were grown in a proprietary greenhouse. Flowers from each plant from each generation were collected, freeze dried and quantitated for carotenoid levels using HPLC-based, industry standard AOAC method. Briefly, 0.5-1 g ground petal samples were extracted and saponified with 30 mL HEAT extraction solvent (hexane, ethanol, acetone, and toluene in volume ratios of 10:6:7:7) and 2 mL 40% methanolic potassium hydroxide (w/v) in a 56° C. water bath set to shake at 120 rpm for 20 minutes. The extract was analyzed in an HPLC with a Waters Sphersorb CN column (50×4.6 mm, 3 m). An isocratic elution with mobile phase of hexanes (74.6%), methylene chloride (24.9%), methanol (0.4%), n,n-diisopropylethylamine (0.1%) at 1.000 mL/min flow rate was used. The column temperature was maintained at 25° C. and 10 μL of sample was injected to run for 13 minutes per assay. The diode array detector (DAD) was set at 446 nm. A standard method was used for the quantitation of lutein and zeaxanthin. An individual F4 plant was identified and selfed to develop, KI3928 F₅ seed. Average PLC showed substantial improvement in each generation during pedigree breeding and selection of progeny. The mean PLC of KI3928 was 22.21±1.14 mg/g on a dry weight basis with upper confidence interval (U-CI) of 22.77 and lower confidence interval (L-CI) of 21.63. This is a moderate increase in PLC in the KI3928 male inbred line, however, the hybrids derived from KI3928 as a male parent showed excellent general combining ability (GCA) for the PLC trait. At each generation visual observation and chemotypic screening and selection methods were performed by breeders to carefully select and advance the progeny. KI3928 was finalized as a homozygous inbred male line with unique assembly of alleles for contribution of higher petal lutein content and double flowers in hybrid combinations.

Petal Lutein Content (PLC) analysis in hybrids derived from KI3928 male parent. Five higher xanthophyll accumulating hybrids, XanHy-1, XanHy-2, XanHy-3, XanHy-4 and XanHy-5 derived from the male inbred parent, KI3928 with five different female parents were evaluated for petal lutein content (PLC) on a dry weight basis. These hybrids were compared for PLC with two control hybrids, CK-1 and CK-2. All seven hybrids were grown in replicated small plot field trials for petal sample collection at multiple harvests in a randomized block design. All five hybrids, XanHy-1 to XanHy-5 had statistically significant higher PLC compared to both controls, CK-1 and CK-2 (Table 1). The CK-1 and CK-2 had mean PLCs of 20.6 mg/g and 22.48 mg/g, respectively, whereas all the five hybrids derived from male parent, KI3928 had PLCs ranging from 23.46 mg/g to 25.58 mg/g (Table 1). The carotenoid profiles of five hybrids derived from the male inbred parent, KI3928 crossed with five female parents were indicators of the degree of stability of the PLC trait in an inbred background of KI3928 across varying female backgrounds in hybrid combination. Similar stability and robustness were also observed for the double flower trait in all five hybrids derived from the male parent KI3928 for flower yield and other agronomic traits. The KI3928 male inbred parent represents a unique assemblage of alleles for PLC, double flowers and flower yield improvements in hybrid combinations.

TABLE 1
Comparison of petal lutein content (PLC) in some of the hybrid
combinations derived from KI3928 male parent with two checks
in multi-year, multi-location hybrid evaluation field trials

| | LS Mean | Mean | | % PLC | | | | | |
|---|---|---|---|---|---|---|---|---|---|
| | PLC | PLC | Tukey's | diff to | | | | | |
| Hybrid | (mg/g) | (mg/g) | HSD | CK-1 | N | SD | CV | L-CI | U-CI |
| XanHy-5 | 25.58 | 25.6 | A | 24 | 47 | 3.1 | 12 | 24.71 | 26.49 |
| XanHy-4 | 25.35 | 25.4 | AB | 23 | 47 | 3.5 | 13.9 | 24.4 | 26.4 |
| XanHy-3 | 24.99 | 25 | AB | 21 | 45 | 3.6 | 14.5 | 23.95 | 26.05 |
| XanHy-2 | 24.26 | 24.3 | BC | 18 | 59 | 3.9 | 16 | 23.3 | 25.3 |
| XanHy-1 | 23.46 | 23.5 | C | 14 | 180 | 3.6 | 15.3 | 22.97 | 24.03 |
| CK-2 | 22.48 | 22.5 | D | 9 | 223 | 3.3 | 14.8 | 22.07 | 22.93 |
| CK-1 | 20.6 | 20.6 | E | 0 | 162 | 2.6 | 12.8 | 20.2 | 21 |

In accordance with one aspect of the present invention provided, is inbred marigold seed and plants thereof, designated KI3928. The present invention further relates to a method for producing inbred marigold seeds that includes, but is not limited to, the steps of planting seed of inbred marigold KI3928 in proximity to itself or to different seed from the same line, growing the resulting marigold plants, cross-pollinating the resulting marigold plants, and harvesting resultant seed obtained from such inbred plants using techniques standard in the agricultural arts such as would be necessary to bulk-up parent seed such as for hybrid production. The present invention also relates to inbred seed produced by such a method.

In any cross between the inbred marigold plant KI3928 and another inbred marigold plant, KI3928 may be designated as the male (pollen parent) or the female (seed parent). The present invention also relates to a marigold plant that expresses substantially all of the physiological and morphological characteristics of inbred the marigold plant KI3928 and to a substantially homogenous population of marigold plants having all the physiological and morphological characteristics of the inbred marigold plant KI3928. Any marigold plants produced from the inbred marigold plant KI3928 are contemplated by the present invention and are, therefore, within the scope of this invention. A description of physiological and morphological characteristics of marigold plant KI3928 is presented in Table 2.

It should be appreciated by one having ordinary skill in the art that, for the quantitative characteristics identified in Table 2, the values presented are typical values. These values may vary due to the environment and accordingly, other values that are substantially equivalent are also within the scope of the invention.

Inbred marigold line KI3928 shows uniformity and stability within the limits of environmental influence for the traits described in Table 2. Inbred KI3928 has been self-pollinated a sufficient number of generations with careful attention paid to uniformity of plant type to ensure the homozygosity and phenotypic stability necessary to use in large scale, commercial hybrid seed production. The KI3928 line has been increased both by hand in isolated fields with continued observations for uniformity. No variant traits have been observed or are expected in KI3928.

The plants of the present invention have the taxonomic description of being genus Tagetes, species erecta, family Asteraceae and the common name marigold. Table 2 describes the traits of the plants through observation of the plants growing in a greenhouse in Hereford, Texas during vegetative and flowering stages, with characteristic traits of KI3928. Table 3 provides descriptions of the traits summarized in Table 2.

TABLE 2
Characteristics of KI3928 at vegetative and flowering stage
Ploidy: Diploid
Chromosome number: 12 sets
Greenhouse Observations

| | Average | | Range | |
|---|---|---|---|---|
| Growth Form | Erect | | | |
| Plant height class (flowering) | Medium | | 46-61 | cm |
| Flowering Season | Mid-Season | | | |
| Flower Type | Chrysanthemum | | | |
| Flower Fullness | Semi-double | | Semi-double | |
| Silhouette | Flattened | | | |
| Number of Flower heads per | 30 | | 25-35 | |
| plant (98 days) | | | | |
| Flower Head Diameter | 6.7 | cm | 6.0-7.5 | cm |
| Flower Odor | Mild “Marigold” | | | |
| Flower Head Color | Medium to Dark | | | |
| | Orange | | | |
| Days from planting to first | 65 | days | 62-68 | days |
| flower | | | | |
| Length of flowering season | 45 | days | 45-55 | days |
| Plant height at maturity | 85 | cm | 70-100 | cm |
| (Flowering) (98 days) | | | | |
| Plant width class (flowering) | 47 | cm | 45-50 | cm |
| Leaf Type | Compound | | | |
| Leaf Shape | Lanceolate | | | |
| Leaf Margin | Dentate | | | |
| Leaf Width | 4.0 | cm | 3.8-5.0 | cm |
| Leaf Length | 12.0 | cm | 10-14 | cm |
| Leaf Color | Green | | | |
| Petiole Anthocyanin | Green | | | |
| Stem Profile | Herbaceous | | | |
| Stem Structure | Intermediate | | | |
| Node Length (middle of plant) | 5 | cm | 4-6 | cm |
| (flowering)(98 days) | | | | |
| Number of internodes below first | 1 | | 1-2 | |
| branch | | | | |
| Number of first order branches | 12.5 | | 10-15 | |
| (flowering)(98 days) | | | | |

TABLE 3
Description of Vegetative and Flowering Characteristics.

| Ploidy | Number of sets of chromosomes |
|---|---|
| Chromosome Number | Number of chromosomes |
| Plant Height (Flowering) | Measure length of the plant from top of soil |
| | media to highest plant growth |
| Plant Height Class | Height comparable with other marigold varieties |
| Growth Form | Plant phenotype |
| Leaf Type | Type of leaf |
| Leaf Shape | Structure of leaf |
| Leaf Margin | Structure of leaf edge |
| Leaf width (cm) | Width of leaf structure from fully developed |
| | leaf from mid-section of plant |
| Leaf Length (cm) | Length of leaf structure from fully developed |
| | leaf from mid-section of plant |
| Leaf Color | Visual color of leaf |
| Petiole Anthocyanin | Red pigmentation of leaf stalk |
| Stem profile | Shape of Stem |
| Stem Structure | Pliability of stem |
| Node length | An internode measured at the mid-section |
| | of the main stalk of the plant |
| Number of nodes below first | Visual number of internodes below first branch |
| branch | |
| Number of first order branches | Number of branches generating from the main stalk |
| Days from planting to first | Observed days between sowing to first open flower |
| flower | |
| Length of flowering season | Observed days between sowing to end of flowering |
| Flowering Season | Designated by number of days to flower |
| | compared to other crops |
| Silhouette | Petal Structure |
| Number of Flower heads per | Number of flowers or buds per plant |
| plant | |
| Flower head diameter | Diameter of stage 3 flower |
| Flower Odor | Strength of flower fragrance |
| Flower Head Color | Petal color |

According to one embodiment, the plant denominated KI3928 produces lutein comprising greater than 26 mg/g on a dry weight petal biomass.

The present invention is related to the development of a novel, stable, inbred line of Tagetes erecta. This line is unique and clearly distinct from all other existing varieties of Tagetes erecta. Line KI3928 has fertile male flowers and is particularly suited for use as a male inbred line which is crossed with female plants to produce hybrid seed that will result in plants having plant tissues, particularly flower petals, that are high in lutein content. The genetic loci present in KI3928 are dominant or partially dominant and can therefore increase the lutein content of a hybrid by up to 50% depending on the female parent of hybrid combination. It is expected that if compatible female inbred parents are being used with KI3928 male inbred parent, the resulting hybrid will also have a lutein content >30 mg/g relative to commercially available hybrids which typically average 20 mg/g lutein on a dry weight basis.

Various breeding schemes may be used to produce new inbred marigold lines from KI3928. In one method, generally referred to as the pedigree method, KI3928 may be crossed with another different marigold plant such as a second inbred parent marigold plant, which either itself exhibits one or more selected desirable characteristic(s) or imparts selected desirable characteristic(s) to a hybrid combination. Examples of potentially desired characteristics include greater flower yield, higher lutein content, reduced time to crop maturity, better agronomic quality, resistance and/or tolerance to insecticides, herbicides, pests, heat and drought, and disease, and uniformity in germination times, stand establishment, growth rate, maturity and flower size. If the two original parent marigold plants do not provide all the desired characteristics, then other sources can be included in the breeding population. Elite inbred lines, that is, pure breeding, homozygous inbred lines, can also be used as starting materials for breeding or source populations from which to develop inbred lines.

Thereafter, resulting seed is harvested and resulting superior progeny plants are selected and selfed or sib-mated in succeeding generations, such as for about 2 to about 10, or more specifically for about 5 to about 7, or alternatively 5 or more generations, until a generation is produced that no longer segregates for substantially all factors for which the inbred parents differ, thereby providing a large number of distinct, pure-breeding inbred lines.

In another embodiment for generating new inbred marigold plants, generally referred to as backcrossing, one or more desired traits may be introduced into inbred parent marigold plant KI3928 (the recurrent parent) by crossing the KI3928 plants with another marigold plant (referred to as the donor or non-recurrent parent) which carries the gene(s) encoding the particular trait(s) of interest to produce F₁ progeny plants. Both dominant and recessive alleles may be transferred by backcrossing. The donor plant may also be an inbred, but in the broadest sense can be a member of any plant variety or population cross-fertile with the recurrent parent. Next, F₁ progeny plants that have the desired trait are selected. Then, the selected progeny plants are crossed with KI3928 to produce backcross progeny plants. Thereafter, backcross progeny plants comprising the desired trait and the physiological and morphological characteristics of marigold inbred line KI3928 are selected. This cycle is repeated for about one to about eight cycles, preferably for about 3 or more times in succession to produce selected higher backcross progeny plants that comprise the desired trait and all of the physiological and morphological characteristics of marigold inbred line KI3928 listed in Table 2 as determined at the 5% significance level when grown in the same environmental conditions. Exemplary desired trait(s) include insect resistance, herbicide resistance, yield stability, yield enhancement and resistance to bacterial, fungal and viral disease. One of ordinary skill in the art of plant breeding would appreciate that a breeder uses various methods to help determine which plants should be selected from the segregating populations and ultimately which inbred lines will be used to develop hybrids for commercialization. In addition to the knowledge of the germplasm and other skills the breeder uses, a part of the selection process is dependent on experimental design coupled with the use of statistical analysis. Experimental design and statistical analysis are used to help determine which plants, which family of plants, and finally which inbred lines and hybrid combinations are significantly better or different for one or more traits of interest. Experimental design methods are used to assess error so that differences between two inbred lines or two hybrid lines can be more accurately determined. Statistical analysis includes the calculation of mean values, determination of the statistical significance of the sources of variation, and the calculation of the appropriate variance components. Either a five or a one percent significance level is customarily used to determine whether a difference that occurs for a given trait is real or due to the environment or experimental error. One of ordinary skill in the art of plant breeding would know how to evaluate the traits of two plant varieties to determine if there is no significant difference between the two traits expressed by those varieties. Mean trait values may be used to determine whether trait differences are significant, and preferably the traits are measured on plants grown under the same environmental conditions.

Of course, the other marigold plant may be the recurrent parent whereby the genetic loci associated with high lutein values in KI3928 are transferred to the recurrent parent following the foregoing process. It is expected that use of such an inbred line in a cross with KI3928 would result in hybrid marigold plants with greater than 40 mg/g lutein on a dry matter basis.

This method results in the generation of inbred marigold plants with substantially all of the desired morphological and physiological characteristics of the recurrent parent and the particular transferred trait(s) of interest. Because such inbred marigold plants are heterozygous for loci controlling the transferred trait(s) of interest, the last backcross generation would subsequently be selfed to provide pure breeding progeny for the transferred trait(s).

It should be appreciated by those having ordinary skill in the art that backcrossing can be combined with pedigree breeding as where inbred KI3928 is crossed with another marigold plant, where the resultant progeny is then back-crossed to inbred KI3928 and thereafter, the resulting progeny of this single backcross are subsequently inbred to develop new inbred lines. This combination of backcrossing and pedigree breeding is useful, for example, when recovery of fewer than all of the KI3928 characteristics than would be obtained by a conventional backcross are desired.

Once inbred lines are created, the next step is to determine if the inbreds have any value in hybrid combinations. This is accomplished by techniques of measuring the combining ability of the new inbred plant, as well as the performance of the line itself. Combining ability refers to a line's contribution as a parent when crossed with other lines to form hybrids. Specific combining ability (SCA) refers to the ability of a line to cross to another specific inbred to form a hybrid. General combining ability (GCA) refers to the ability of a line to cross to a wide range of lines to form hybrids. GCA and SCA of parents in hybrids are measured for specific trait of interest to commercial values. The methodology of forming hybrids to evaluate an inbred line's contribution as a parent for the purpose of selecting superior lines is interchangeably known as experimental, top or test crossing.

In accordance with processes of the present invention, a hybrid plant having inbred KI3928 as a parent is crossed with itself or any different marigold plant such as an inbred marigold plant or a hybrid marigold plant to develop a novel inbred line. For example, a hybrid marigold plant having inbred marigold plant KI3928 as a parent may be inbred, i.e., crossed to itself or sib-pollinated, and the resulting progeny each selfed for about 5 to about 7 or more generations, thereby providing a set of distinct, pure-breeding inbred lines wherein each of the lines received all of its alleles from the hybrid marigold plant having inbred marigold plant KI3928 as a parent. In other embodiments, a hybrid marigold plant having inbred marigold plant KI3928 as a parent is crossed with a different marigold plant that may include any inbred marigold plant that is not inbred marigold plant KI3928, any hybrid marigold plant that does not have KI3928 as a parent, another germplasm source, a mutation inducing stock, or a trait donor plant, thereby providing a set of distinct, pure-breeding inbred lines. The resulting inbred lines could then be crossed with other inbred or non-inbred lines and the resulting inbred progeny analyzed for beneficial characteristics. In this way, novel inbred lines conferring desirable characteristics could be identified.

In yet another aspect of the invention, the present invention relates to processes for producing marigold seeds or plants, where the processes generally comprise crossing a first parent marigold plant with a second parent marigold plant wherein at least one of the first parent marigold plant or the second parent marigold plant is inbred parent marigold plant KI3928. In some embodiments of the present invention, the first inbred marigold plant is KI3928 and is a female and in other embodiments the first inbred marigold plant is KI3928 and is a male. These processes may be further exemplified as processes for preparing hybrid marigold seed or plants, wherein a first inbred marigold plant is crossed with a second marigold plant of a different, distinct variety to provide a hybrid that has, as one of its parents, the inbred marigold plant line KI3928. In this case, a second inbred line is selected which confers desirable characteristics when in hybrid combination with the first inbred line. In these processes, crossing will result in the production of seed. The seed production occurs regardless of whether the seed is collected.

Any time the inbred marigold plant KI3928 is crossed with another, different marigold inbred, a first generation (F₁) marigold hybrid plant is produced. As such, an F₁ hybrid marigold plant may be produced by crossing KI3928 with any second inbred marigold plant. Therefore, any F₁ hybrid marigold plant or marigold seed which is produced with KI3928 as a parent is part of the present invention.

When inbred marigold plant KI3928 is crossed with another inbred plant to yield a hybrid, the original inbred can serve as either the maternal or paternal plant with basically, the same characteristics in the hybrids. Occasionally, maternally inherited characteristics may express differently depending on the decision of which parent to use as the female. However, often one of the parental plants is preferred as the maternal plant because of increased lutein yield and preferred production characteristics. It is generally preferable to use KI3928 as the male parent.

For a decision to be made to advance a hybrid, it is not necessary that the hybrid be better than all other hybrids. Rather, significant improvements must be shown in at least some traits that would create value for some applications or markets. Some testcross hybrids are eliminated despite being similarly competitive relative to the current commercial hybrids because of the cost to bring a new hybrid to market requires a new product to be a significant improvement over the existing product offering.

Another aspect of the present invention relates to plants produced using inbred marigold plant KI3928, where all plants produced using KI3928 as a parent are within the scope of this invention, including plants derived from inbred marigold plant KI3928. This includes plants essentially derived from inbred KI3928 with the term “essentially derived variety” having the meaning ascribed to such term in 7 U.S.C. § 2104(a)(3) of the Plant Variety Protection Act, which definition is hereby incorporated by reference. This also includes progeny plant and parts thereof with at least one ancestor that is inbred marigold plant KI3928 and more specifically where the pedigree of this progeny includes 1, 2, 3, 4, and/or 5 or cross pollinations to inbred marigold plant KI3928, or a plant that has KI3928 as a progenitor. All breeders of ordinary skill in the art maintain pedigree records of their breeding programs. These pedigree records contain a detailed description of the breeding process, including a listing of all parental lines used in the breeding process and information on how such lines were used. Thus, a breeder would know if KI3928 were used in the development of a progeny line and would also know how many breeding crosses to a line other than KI3928 were made in the development of any progeny line. A progeny line so developed may then be used in crosses with other, different, marigold inbreds to produce first generation (F₁) marigold hybrid seeds and plants with superior characteristics.

Accordingly, another aspect of the present invention relates to methods for producing an inbred marigold line KI3928-derived marigold plant. This method for producing a KI3928-derived marigold plant, comprises: (a) crossing inbred marigold plant KI3928 with a second marigold plant to yield progeny marigold seed; and (b) growing the progeny marigold seed, (under plant growth conditions), to yield the KI3928-derived marigold plant. Such methods may further comprise the steps of: (c) crossing the KI3928-derived marigold plant with itself or another marigold plant to yield additional KI3928-derived progeny marigold seed; (d) growing the progeny marigold seed of step (b) (under plant growing conditions), to yield additional KI3928-derived marigold plants; and (e) repeating the crossing and growing steps of (c) and (d) from between 0 to 7 times, or between 0 to 10 times, or more, to generate further KI3928-derived marigold plants. Still further, this may comprise utilizing methods of haploid breeding and plant tissue culture methods to derive progeny of the KI3928-derived marigold plant.

With the advent of molecular biological techniques that have allowed the isolation and characterization of genes that encode specific protein products, scientists in the field of plant biology developed a strong interest in engineering the genome of plants to contain and to express foreign genes, or additional, or modified versions of native or endogenous genes (perhaps driven by different promoters) to alter the traits of a plant in a specific manner. Such foreign, additional and/or modified genes are referred to herein collectively as “transgenes.” The present invention, in particular embodiments, also relates to transformed versions of the claimed inbred marigold line KI3928 containing one or more transgenes, particularly genes that encode resistance to an herbicide.

The present invention, in particular embodiments, also relates to genetically engineered KI3928 and its progeny. For instance, in at least one embodiment, genome editing technologies, including but not limited to those based on CRISPR/Cas9, are used to create a genetically-engineered marigold line KI3928 and its derived progeny. The present invention, in at least one embodiment, relates to a genetically engineered marigold plant, or parts thereof, or progeny marigold seed, containing one or more desired traits. In preferred embodiments, the resulting plant line is substantially similar to marigold line KI3928, but includes genetically-engineered genes or desired traits. By way of non-limiting example, desired traits may include herbicide and/or pesticide tolerance, where the event sequence may be stacked with other traits, for instance those with identified herbicide or pesticide tolerant genes and/or proteins.

Numerous methods for plant transformation have been developed, including biological and physical, plant transformation protocols, the disclosures of which are expressly incorporated in their entirety herein. See, e.g., Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, e.g., Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

The foregoing methods for transformation would typically be used for producing transgenic inbred lines. Transgenic inbred lines could then be crossed, with another (non-transformed or transformed) inbred line, in order to produce a transgenic hybrid marigold plant. Alternatively, a genetic trait which has been engineered into a particular marigold line using the foregoing transformation techniques could be moved into another line using traditional backcrossing techniques that are well known in the plant breeding arts. For example, a backcrossing approach could be used to move an engineered trait from a public, non-elite line into an elite line, or from a hybrid marigold plant containing a foreign gene in its genome into a line or lines which do not contain that gene.

In addition to phenotypic observations, a plant can also be described by its genotype. The genotype of a plant can be described through a genetic marker profile which can identify plants of the same variety, a related variety or be used to determine or to validate a pedigree. Genetic marker profiles can be obtained by techniques such as Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as Microsatellites, Isozyme Electrophoresis, Isolelectric Focusing and, the latest maker, Single Nucleotide Polymorphisms (SNPs).

Particular markers used for these purposes are not limited to the set of markers disclosed herewithin but are envisioned to include any type of genetically stable marker and marker profile which provides a means of distinguishing varieties. In addition to being used for identification of inbred parents, a hybrid produced through the use of KI3928 or its parents, and identification or verification of the pedigree of progeny plants produced through the use of KI3928, the genetic marker profile is also useful in breeding and developing backcross conversions.

### DEPOSIT INFORMATION

Applicants have made a deposit of at least 2,500 seeds of inbred Tagetes erecta line KI3928 with the American Type Culture Collection (ATCC), 10801 University Boulevard, Manassas, Va. 20110 USA, under ATCC Accession No. PTA-127534. The seeds received by the ATCC on Feb. 22, 2023, were taken from a deposit maintained by Kemin Industries, Inc. since prior to the filing date of this application. Access to this deposit will be available during the pendency of the application to the Commissioner of Patents and Trademarks and persons determined by the Commissioner to be entitled thereto upon request. Upon allowance of any claims in the application, the Applicant will make the deposit available to the public pursuant to 37 C.F.R. § 1.808. This deposit of the Marigold Inbred Line KI3928 will be maintained in the ATCC depository, which is a public depository, for a period of 30 years, or 5 years after the most recent request, or for the enforceable life of the patent, whichever is longer, and will be replaced if it becomes nonviable during that period. Additionally, Applicant has or will satisfy all of the requirements of 37 C.F.R. §§ 1.801-1.809, including providing an indication of the viability of the sample upon deposit. Applicant has no authority to waive any restrictions imposed by law on the transfer of biological material or its transportation in commerce.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

We claim:

1. Seed of marigold inbred line designated KI3928, or a part of the seed, representative seed of the line having been deposited under ATCC Accession No. PTA-127534.

2. A substantially homogenous population of seeds comprising at least one of the marigold seed of claim 1.

3. A method for producing marigold seed, said method comprising the steps of:
(a) planting seed of claim 1 in proximity to another seed of inbred line KI3928 or to a seed of a different line;
(b) growing plants from the seed;
(c) pollinating said plants; and
(d) harvesting resultant seed.

4. A marigold seed produced by the method of claim 3.

5. A marigold plant produced by growing the seed of claim 1.

6. A part of the marigold plant of claim 5, selected from the group consisting of an intact plant cell, a plant protoplast, an embryo, pollen, an ovule, a flower, a seed, a flower, a petal and a leaf.

7. Pollen of the plant of claim 5.

8. An ovule of the plant of claim 5.

9. A marigold plant, or a part of the marigold plant, having all physiological and morphological characteristics of the marigold plant, or said part of the marigold plant, of claim 5.

10. A substantially homogenous population comprising at least one of the marigold plants of claim 5.

11. A method for producing a marigold plant, said method comprising the step of: (a) crossing inbred marigold plant KI3928, representative seed of the line having been deposited under ATCC Accession No. PTA-127534, with another marigold line plant to yield progeny marigold seed.

12. The method of claim 11, wherein the said other marigold line plant is an inbred marigold plant.

13. The method of claim 11, further comprising the steps of: (b) growing the progeny marigold seed from step (a) under self-pollinating or sib-pollinating conditions for at least 5 generations; and (c) harvesting resultant seed.

14. The method of claim 11, further comprising the step of selecting plants obtained from growing at least one generation of the progeny marigold seed for a desirable trait.

15. A method of introducing a desired trait from marigold inbred line KI3928, representative seed of the line having been deposited under ATCC Accession No. PTA-127534, said method comprising the steps of:
(a) crossing KI3928 plants with plants of another marigold line that comprise a desired trait to produce F1 progeny plants;
(b) selecting F1 progeny plants that have the desired trait;
(c) crossing selected progeny plants with said other marigold line plants to produce backcross progeny plants;
(d) selecting for backcross progeny plants that comprise the desired trait and physiological and morphological characteristics of marigold inbred line KI3928; and
(e) performing steps (c) and (d) one or more times in succession to produce the selected or higher backcross progeny plants that comprise the desired trait and all of the physiological and morphological characteristics of said other marigold inbred line as determined at the 5% significance level when grown in the same environmental conditions.

16. A method for producing a hybrid marigold seed comprising crossing a first inbred parent marigold plant with a second inbred parent marigold plant and harvesting resultant hybrid marigold seed, wherein the first inbred marigold plant or the second inbred marigold plant is the marigold plant of claim 5.

17. A method for producing a hybrid marigold seed, the method comprising:
(a) planting in pollinating proximity a seed of the inbred marigold plant of claim 5 and a seed of a second, different inbred marigold plant;
(b) cultivating the seeds into plants that bear flowers;
(c) cross pollinating at least some of the cultivated plants; and
(d) harvesting seeds produced from at least one of the first or second inbred marigold plants.

18. The method according to claim 17, wherein the KI3928 marigold plant is the male parent.

19. The method according to claim 17, wherein the KI3928 marigold plant is the female parent.

20. A hybrid marigold seed produced by the method according to claim 17.

21. A hybrid marigold plant, or parts thereof, produced by growing the hybrid marigold seed of claim 20.

22. A genetically engineered marigold plant, or parts thereof, or progeny marigold seed, containing one or more desired trait.
